(19) United States
(12) Patent Application Publication
(10) Pub. No.: US 2021/0040116 A1
(43) Pub. Date: Feb. 11, 2021

(54) New Polymorphs of Dolutegravir Sodium

(71) Applicant: CIPLA LIMITED, Mumbai (IN)

(72) Inventors: Dharmaraj Ramachandra RAO, Thane (West) (IN); Geena MALHOTRA, Mumbai (IN); Manjinder Singh PHULL, Mumbai (IN); Ashwini Amol SAWANT, Mumbai (IN); Amit NAIK, Navi Mumbai (IN); Manish Gopaldas GANGRADE, Mumbai (IN); Shashirekha KANATHALA, Navi Mumbai (IN)

(21) Appl. No.: 16/644,793

(22) PCT Filed: Sep. 7, 2017
(86) PCT No.: PCT/GB2017/052621

§ 371 Date: Mar. 5, 2020

(51) Int. Cl. C07D 498/14

(52) U.S. Cl. CPC C07D 498/14; C07B 2200/13

(57) ABSTRACT

The present invention relates to new polymorphs of dolutegravir sodium and processes for the preparation of the new polymorphs of dolutegravir sodium. The present invention also provides pharmaceutical compositions comprising these new polymorphs, and medical uses of the polymorphs.

### CROSS REFERENCE TO RELATED APPLICATIONS

This Application is a filing under 35 U.S.C. 371 of International Application No. PCT/GB2017/052621 filed Sep. 7, 2017, entitled “New Polymorphs of Dolutegravir Sodium,” which is incorporated by reference herein in its entirety.

### TECHNICAL FIELD OF THE INVENTION

The present invention relates to new polymorphs of dolutegravir sodium and processes for the preparation of the new polymorphs of dolutegravir sodium. And pharmaceutical compositions containing these new polymorphs.

### BACKGROUND

Dolutegravir sodium is referred to as second generation integrase inhibitor, chemically known as sodium (4R,12aS)-9-{[(2,4,-difluorophenyl)methyl]carbamoyl}-4-methyl-6,8-dioxo-3,4,6,8,12,12a-hexahydro-2H-pyrido[1′,2′:4,5]pyrazino[2,1-b][1,3]oxazin-7-olate (also known as (IUPAC): sodium; (4R,12aS)-9-[(2,4-difluorophenyl)methylcarbamoyl]-4-methyl-6,8-dioxo-3,4,12,12a-tetrahydro-2H-pyrido[5,6]pyrazino[2,6-b][1,3]oxazin-7-olate). The structure of dolutegravir sodium is represented as follows:

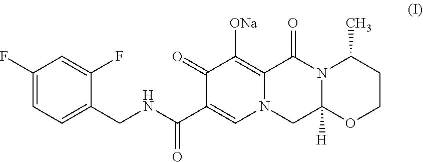

Dolutegravir sodium is indicated in combination with other antiretroviral agents for the treatment of human immunodeficiency virus type 1 (HIV-1) infection.

Dolutegravir sodium and its preparation was first disclosed in WO2006116764. WO2010068253 describes preparation of dolutegravir sodium and other compounds. It also describes crystalline forms of dolutegravir and dolutegravir sodium characterised by X-ray diffraction (XRD), infrared spectroscopy (IR) and carbon NMR spectroscopy (¹³C NMR). WO2010068253 and U.S. Pat. No. 9,242,986 disclose a crystal form of a sodium salt of a compound of formula AA having characteristic X-ray diffraction peaks at 6.4°±0.2°, 9.2°±0.2°, 13.8°±0.2°, 19.2°±0.2° and 21.8°±0.2° 2θ in an X-ray powder diffraction pattern. WO2010068253 and U.S. Pat. No. 9,242,986 also disclose a crystal form of a hydrate of a sodium salt of a compound of formula AA having characteristic X-ray diffraction peaks at 8.0°±0.2°, 9.3°±0.2°, 11.3°±0.2°, 16.0°±0.2°, and 22.8°±0.2° 2θ in an X-ray powder diffraction pattern.

WO2013038407 describes amorphous form of dolutegravir sodium and preparation thereof.

WO2015092752 describes form M1 while WO2015118460 describes forms M2, M3 and M4 of dolutegravir sodium. WO2015118460 refers to the crystal form of (2) having characteristic X-ray diffraction peaks at 6.4°±0.2°, 9.2°±0.2°, 13.8°±0.2°, 19.2°±0.2° and 21.8°±0.2° 2θ disclosed in WO2010068253 as “a crystalline form of dolutegravir sodium”. WO2015118460 also refers to the crystal form of (2) having characteristic X-ray diffraction peaks at 8.0°±0.2°, 9.3°±0.2°, 11.3°±0.2°, 16.0°±0.2°, and 22.8°±0.2° 2θ disclosed in WO2010068253 as “a crystalline form of dolutegravir sodium hydrate”. We adopt the same naming used by WO2015118460 for the crystalline forms disclosed in WO2010068253 when discussing our comparative intrinsic dissolution experiments.

WO2015138933 describes dolutegravir sodium Forms II, III, IV, V, VI, VII, VIII, IX, X and XI.

WO2015139591 describes polymorphic forms A, B, C, D, E of dolutegravir sodium.

While WO2015009927, WO2015110897 and WO2015111080 describe various salts such as potassium and calcium of dolutegravir.

It is a well known fact that different polymorphic forms of the same drug may have substantial differences in certain pharmaceutically important properties such as dissolution characteristics, bioavailability patterns, handling properties, solubility, flow characteristics and stability.

Further, different physical forms may have different particle size, hardness and glass transition temperatures.

These physical characteristics are influenced by the conformation and orientation of the molecule in the unit cell which are different for different polymorphic forms.

And these polymorphic forms exhibit distinct X-ray diffractogram, solid state ¹³C NMR spectrometry, infrared spectrometry. Further, these polymorphic forms may give rise to peculiar thermal behaviour which can be measured by melting point, thermogravimetric analysis (TGA), differential scanning calorimetry (DSC). All these properties can be used to distinguish a particular polymorph from the other form.

### OBJECTIVES OF THE INVENTION

The object of the invention is to provide new polymorphic forms of dolutegravir sodium. And processes for the preparation of these new polymorphic forms of dolutegravir sodium.

Another object of the present invention is to provide pharmaceutical compositions comprising new polymorphic forms of dolutegravir sodium.

### SUMMARY OF THE INVENTION

In one aspect, the invention provides new polymorphic forms which hereinafter are referred to as crystalline form (also known as “polymorph”) 1A and form 1B of dolutegravir sodium.

In another aspect, the invention provides processes for the preparation of new polymorphic forms of dolutegravir sodium.

In yet another aspect, the invention comprises pharmaceutical compositions comprising new polymorphic forms of dolutegravir sodium along with a pharmaceutically acceptable carrier.

In an aspect, there is provided crystalline polymorph 1A of dolutegravir sodium. The polymorph 1A may be characterised by having an X-ray powder diffraction pattern, in terms of degrees 2-theta, at 15.66°, 20.29° and 22.51°±0.2° 2 theta. The XRPD may further comprise two peaks between 5.75° and 6.55°±0.2° 2 theta. The XRPD may include yet further peaks at 8.00° and 25.53°±0.2° 2 theta. The XRPD may also include yet further peaks at 18.36°, 21.11°, and 23.49°±0.2° 2 theta. The XRPD may still include yet further peaks at 8.76°, 9.68°, 10.95°, 11.94°, 12.74°, 13.15°, 17.00°, 19.84°, 26.72° and 27.48°±0.2° 2 theta.

Polymorph 1A may be characterised by powder X-ray diffraction pattern, as shown in FIG. 1.

Polymorph 1A may be characterised by IR spectrum, as shown in FIG. 3.

In an aspect, there is provided crystalline polymorph 1A of dolutegravir sodium having a bulk density ranging from 0.3-0.6 g/ml, preferably ranging from 0.4-0.5 g/ml.

In an aspect, there is provided crystalline polymorph 1A of dolutegravir sodium having a tapped density ranging from 0.6-0.9 g/ml, preferably from 0.7-0.8 g/ml.

It would be clear to the skilled person that crystalline polymorph 1A of dolutegravir sodium may be characterised by one or more of the above characteristics.

In an aspect, there is provided a process for preparing crystalline polymorph 1A of dolutegravir sodium as defined in any preceding statement, the process comprising the steps of:
a) preparing a mixture of dolutegravir base with methanol,
b) cooling the reaction mixture from step a) to a temperature ranging from −10° C. to 20° C.
c) preparing a solution of sodium hydroxide in a solvent,
d) mixing the solutions from steps b) and c) over a period of time less than 60 minutes at a temperature ranging from −10° C. to 20° C.,
e) maintaining the reaction mass from step d) at a temperature in the range of −10° C. to 20° C. over a period of time ranging from 30 minutes to 3 hours, and
f) isolating crystalline polymorph 1A of dolutegravir sodium.

Further disclosure relating to Form 1A and processes for preparing it may be found in the statements below.

In an aspect, there is provided crystalline polymorph 1B of dolutegravir sodium. The polymorph 1B may be characterised by having an X-ray powder diffraction pattern comprising peaks, in terms of degrees 2-theta, at 7.08°, 10.82°, 12.95°, 15.93° and 18.59°±0.2° 2 theta. The XRPD may include further peaks at 13.82°, 17.44°, 20.09°, 22.78°, 25.41°, 26.35°, 28.81°±0.2° 2 theta.

Polymorph 1B may be characterised by powder X-ray diffraction pattern, as shown in FIG. 2.

Polymorph 1B may be characterised by IR spectrum, as shown in FIG. 4.

It would be clear to the skilled person that crystalline polymorph 1B of dolutegravir sodium may be characterised by one or more of the above characteristics.

In an aspect, there is provided a process for preparing crystalline polymorph 1B of dolutegravir sodium as defined in any one of claims 14 to 16, the process comprising the steps of:
a) preparing a mixture of dolutegravir base with ethanol,
b) cooling the reaction mixture from step a) to a temperature ranging from −10° C. to 20° C.,
c) preparing a solution of sodium hydroxide in a solvent,
d) mixing the solutions from steps b) and c) over a period of time ranging from 30 to 60 minutes at a temperature ranging from −10° C. to 20° C.,
e) maintaining the reaction mass from step d) at a temperature in the range of −10° C. to 20° C. over a period of time ranging from 30 minutes to 3 hours, and
f) isolating crystalline polymorph 1B of dolutegravir sodium.

Further disclosure relating to Form 1B and processes for preparing it may be found in the statements below.

In an aspect, there is provided crystalline form 1A of dolutegravir sodium prepared according to any one of the processes disclosed herein.

In an aspect, there is provided crystalline form 1B of dolutegravir sodium prepared according to any one of the processes disclosed herein.

In an aspect, there is provided a pharmaceutical composition comprising crystalline Form 1A of dolutegravir sodium as disclosed herein together with one or more pharmaceutically acceptable excipients.

In an aspect, there is provided a pharmaceutical composition comprising crystalline Form 1B of dolutegravir sodium as disclosed herein together with one or more pharmaceutically acceptable excipients.

In an aspect, there is provided a pharmaceutical composition comprising crystalline Form 1A of dolutegravir sodium as disclosed herein in combination with one or more further active pharmaceutical ingredients together with one or more pharmaceutically acceptable excipients.

In an aspect, there is provided a pharmaceutical composition comprising crystalline Form 1B of dolutegravir sodium as disclosed herein in combination with one or more further active pharmaceutical ingredients, together with one or more pharmaceutically acceptable excipients.

In an aspect, there is provided a pharmaceutical composition as disclosed herein wherein the dosage form is selected from a list consisting of tablets, capsules, pills, caplets, lozenges, dispersible granules, dry powder syrup, ready to use suspension, parenteral dosage forms, various inhalation formulations, and transdermal formulations.

In an aspect, there is provided a method of treating HIV infection, AIDS and related conditions in a patient, said method comprising administering to the patient a pharmaceutical formulation comprising a therapeutically effective amount of crystalline Form 1A of dolutegravir sodium as disclosed herein.

In an aspect, there is provided a method of treating HIV infection, AIDS and related conditions in a patient, said method comprising administering to the patient a pharmaceutical formulation comprising a therapeutically effective amount of crystalline Form 1B of dolutegravir sodium as disclosed herein.

In an aspect, there is provided a crystalline Form 1A of dolutegravir sodium as disclosed herein for use in treating HIV infection, AIDS and related conditions in a patient.

In an aspect, there is provided a crystalline Form 1B of dolutegravir sodium as disclosed herein for use in treating HIV infection, AIDS and related conditions in a patient.

In an aspect there is provided a use of crystalline Form 1A of dolutegravir sodium as disclosed herein in the manufacture of a medicament for treating HIV infection, AIDS and related conditions in a patient.

In an aspect, there is provided a use of crystalline Form 1B of dolutegravir sodium as disclosed herein in the manufacture of a medicament for treating HIV infection, AIDS and related conditions in a patient.

In an aspect, there is provided a crystalline Form 1A of dolutegravir sodium substantially as herein defined with reference to FIG. 1 or 3.

In an aspect, there is provided a crystalline Form 1B of dolutegravir sodium substantially as herein defined with reference to FIG. 2 or 4.

### DETAILED DESCRIPTION OF THE INVENTION

The present invention provides new polymorphic form 1A and form 1B of dolutegravir sodium.

Crystalline polymorphs may be characterised using methods such as powder X-ray diffraction pattern, infrared spectrum. Those skilled in the art will appreciate that XRD peak positions may vary within a margin of error depending on which apparatus is used to analyse a sample, sample preparation techniques, the calibration techniques applied to the instruments, human operational variation, etc. Those skilled in the art will recognise that the appropriate error of margins for a XRD can be ±0.2°2θ.

Crystalline Polymorphs 1A and 1B can also be characterised by reference to their onset of melting in differential scanning calorimetry (DSC), thermogravimetric analysis (TGA).

The X-ray diffraction data was obtained using Rigaku Ultima IV X-ray powder diffractometer using a Cu K_{α} radiation source. Parameters of the machine include:

Source: Cu Kα

Wavelength: ˜1.5405 A°

Voltage: 40 KV

Current: 30 mA

Scan axis: θ/2 θa

Measurement method: Continuous

Scanning range: 3.0° to 40° 2θ

Goniometer speed: 2.0° 2θ/min.

Sampling width: 0.02° 2θ

Divergence slit: 2/3°

Receiving slit: 0.3 mm

Counting unit: CPS

Detector type: Scintillation Counter

Polymorph 1A may have an X-ray diffraction pattern containing peaks at 5.89, 8.00, 8.76, 9.68, 10.95, 11.94, 12.74, 13.15, 15.66, 17.00, 18.36, 19.84, 20.29, 21.11, 22.51, 23.49, 25.53, 26.72, 27.48±0.2° 2θ.

The XRD pattern of polymorph 1A may be as shown in FIG. 1.

Polymorph 1B may have an X-ray diffraction pattern containing peaks at 7.08, 10.82, 12.95, 13.82, 15.93, 17.44, 18.59, 20.09, 22.78, 25.41, 26.35, 28.81±0.2° 2θ.

The XRD pattern of polymorph 1B may be as shown in FIG. 2.

The IR experiments were performed on a Bruker Alpha FTIR using ATR assembly instrument.

The polymorph 1A may have a FTIR spectrum with characteristic absorption hands at about 1623, 1537, 1500, 1465, 1447, 1421, 1398, 1360, 1321, 1276, 1255, 1237, 1200, 1137, 1093, 1063, 1030, 1020, 963, 921, 882, 863, 841, 816, 790, 759, 741, 722, 685, 664±2 cm⁻¹.

Polymorph 1A may be characterised by IR pattern, as shown in FIG. 3.

The polymorph 1B may have a FTIR spectrum with characteristic absorption bands at about 1658, 1633, 1588, 1531, 1500, 1473, 1450, 1430, 1403, 1364, 1316, 1277, 1251, 1201, 1168, 1136, 1109, 1097, 1089, 1071, 1045, 1021, 984, 962, 920, 907, 880, 859, 813, 787, 766, 745, 722, 686±2 cm⁻¹.

Polymorph 1B may be characterised by IR pattern, as shown in FIG. 4.

Further, these new polymorphs may be characterised by differential scanning calorimetry (DSC), thermogravimetric analysis (TGA).

It should be noted that different equipments and/or conditions may provide slightly different data. Hence, the figures provided should not be taken as absolute.

The invention further provides preparation of polymorphs 1A and 1B of dolutegravir sodium.

The process may involve crystallization or recrystallization of dolutegravir sodium. Optionally, seeding may also be used.

Further, the crystallization may be carried out directly from the reaction mixture or from a subsequent solution or solid.

Various polymorphs of dolutegravir sodium are known to be prepared by first mixing dolutegravir with solvent at room temperature or at reflux temperature and adding sodium hydroxide solution. The reaction mixture is then stirred or kept at room temperature or at higher temperature for the time ranging from 2 hours to 20 hours to get the salt or polymorph.

The inventors of the present invention, prepared the salt at temperatures lower than room temperature and they have surprisingly obtained these new polymorphs. This new process forms another aspect of the present invention.

Dolutegravir sodium form 1A may be prepared by first mixing dolutegravir base with suitable solvent at a suitable temperature. The dolutegravir base may be prepared according to any known prior art process.

The suitable solvent used is selected from the group consisting of C₁₋₄ alcohols, halogenated hydrocarbons or mixtures thereof.

The solvent may be selected from methanol, ethanol, isopropanol, n-butanol, isobutanol, methylene dichloride (MDC), toluene, preferably methanol.

The suitable temperature of mixing in first step ranges from 20° to 35° C., preferably ranging from 22° to 33° C., more preferably ranging from 25° to 30° C.

This reaction mixture may be cooled to a temperature ranging from −10° to 20° C., preferably ranging from −10° to 18° C., more preferably ranging from −10° to 15° C.

Surprisingly present inventors have found this specific temperature range wherein new form 1A is formed. Any deviation from this temperature range may result in the formation of a mixture of polymorphs or the formation of another polymorph. Thus, it is important to maintain this temperature range to get a pure polymorphic form.

Pure polymorphic form means a polymorph which is substantially free of any other polymorphic form, wherein “substantially free of any other polymorphic form” means the polymorph consists of at least 95% of the particular polymorphic form, in some instances the polymorph consists of at least 98% of the particular polymorphic form, preferably at least 98.5%, more preferably at least 99%, yet more preferably at least 99.5%. In some instances the polymorph consists of 100% of the particular polymorphic form i.e. there is no other polymorphic form present.

To the cooled reaction mixture, sodium hydroxide solution may be added over a specific period of time. Sodium hydroxide solution may be made by adding or dissolving sodium hydroxide in a suitable solvent. The suitable solvent used may be selected from the group consisting of water, C₁₋₄ alcohols or mixture thereof. The solvent, C₁₋₄ alcohols, may be selected from methanol, ethanol, isopropanol, n-butanol, isobutanol.

The amount of sodium hydroxide used may range from 1.1 to 2.1 moles with respect to starting material that is dolutegravir base or hydroxyprotected dolutegravir (i.e. dolutegravir with the hydroxy group at the 6-position being protected by methoxy).

The addition of sodium hydroxide solution to dolutegravir solution or vice versa may be carried out in less than 60 minutes, preferably between 30 and 60 minutes. The time period may be around 30 minutes or less than 30 minutes. This time of addition is also very crucial for formation of polymorph because an increase in this time of addition may result the formation of a mixture of polymorphs or the formation of another polymorph. Suitably, the sodium hydroxide solution may be added to the dolutegravir solution, or the dolutegravir solution may be added to the sodium hydroxide solution.

The reaction mass may be maintained at a temperature ranging from −10° to 20° C., preferably ranging from −10° to 15° C. and for specific period of time.

The reaction mass may be maintained for 30 minutes to 3 hours.

Maintaining the reaction mixture at the lower temperature is also important for formation of the desired polymorph.

Finally the reaction mass may be filtered and the product obtained dried.

Alternatively polymorph 1A may directly be obtained from hydroxy protected dolutegravir which is chemically known as (3S,11aR)-N-[(2,4-difluorophenyl)methyl]-3-methyl-6-(methyloxy)-5,7-dioxo-2,3,5,7,11,11a-hexahydro[1,3]oxazolo[3,2-a]pyrido[1,2-d]-pyrazine-8-carboxamide, wherein hydroxy protected dolutegravir is deprotected using processes known in the art to get dolutegravir base. Suitably, dolutegravir base may be isolated before being converted to polymorph C, wherein isolated means that solid dolutegravir base is obtained from the deprotection reaction mass before being used in the process for preparing Form 1A (i.e. in step a) in the claimed process). Suitably, dolutegravir base may be converted to polymorph 1A in situ, wherein in situ means that the dolutegravir base is present in a solution and is not isolated before being used in the process for preparing Form 1A (i.e. in step a) in the claimed process). Suitable solvent may be added to this dolutegravir base which is present in the solution.

The suitable solvent may be selected from the group consisting of C₁₋₄ alcohols, halogenated hydrocarbons or mixtures thereof.

The solvent may be selected from methanol, ethanol, isopropanol, n-butanol, isobutanol, methylene dichloride (MDC), toluene, preferably methanol.

This reaction mixture may be cooled to a temperature ranging from −10° to 20° C., preferably ranging from −10° to 15° C.

Cooling at this specific temperature range is necessary to obtain new form 1A. Any deviation from this temperature range may result in the formation of a mixture of polymorphs or the formation of another polymorph. Thus, it is important to maintain this temperature range to get a pure polymorphic form.

As noted above, pure polymorphic form means a polymorph which is substantially free of any other polymorphic form, wherein “substantially free of any other polymorphic form” means the polymorph consists of at least 95% of the particular polymorphic form, in some instances the polymorph consists of at least 98% of the particular polymorphic form, preferably at least 98.5%, more preferably at least 99%, yet more preferably at least 99.5%. In some instances the polymorph consists of 100% of the particular polymorphic form i.e. there is no other polymorphic form present.

To the cooled reaction mixture, sodium hydroxide solution may be added over a specific period of time. Sodium hydroxide solution may be first made by adding or dissolving sodium hydroxide in a suitable solvent.

The suitable solvent used may be selected from the group consisting of water, C₁₋₄ alcohols or mixture thereof. The solvent, C₁₋₄ alcohols, may be selected methanol, ethanol, isopropanol, n-butanol, isobutanol.

The amount of sodium hydroxide used may range from 1.1 to 2.1 moles with respect to starting material that is dolutegravir base or hydroxyprotected dolutegravir.

The addition of sodium hydroxide solution to dolutegravir solution or vice versa may be carried out in less than 60 minutes, preferably between 30 and 60 minutes. The time period may be around 30 minutes or less than 30 minutes. This time of addition is also very crucial for formation of polymorph because with increase in this time of addition, there is possibility of formation of the mixture of polymorphs or other polymorph. Suitably, the sodium hydroxide solution may be added to the dolutegravir solution, or the dolutegravir solution may be added to the sodium hydroxide solution.

The reaction mass may be maintained at a temperature ranging from −10° to 20° C., preferably ranging from −10° to 15° C. and for a specific period of time.

The reaction mass may be maintained for 30 minutes to 3 hours.

Maintaining the reaction mixture at the lower temperature is also important for formation of the desired polymorph.

Finally the reaction mass may be filtered and the product obtained dried.

Dolutegravir sodium form 1B may be prepared by first mixing dolutegravir base with suitable solvent at suitable temperature. The dolutegravir base may be prepared according to any known prior art process.

The suitable solvent may be selected from the group consisting of C₁₋₄ alcohol, halogenated hydrocarbons or mixtures thereof. The solvent may be selected from methanol, ethanol, isopropanol, n-butanol, isobutanol, methylene dichloride (MDC), toluene, preferably ethanol.

The suitable temperature of mixing in first step ranges from 20° to 35° C., preferably ranging from 25° to 30° C.

This reaction mixture may be cooled to a temperature ranging from −10° to 20° C., preferably ranging from −10° to 15° C.

Surprisingly present inventors have found this specific temperature range wherein form 1B is formed. So this temperature range works for form 1B as well. If the temperature deviates from this temperature range, there is the possibility of the formation of a mixture of polymorphs or the formation of another polymorph. Thus, it is important to maintain this temperature range to get the pure polymorphic form.

As noted above, pure polymorphic form means a polymorph which is substantially free of any other polymorphic form, wherein “substantially free of any other polymorphic form” means the polymorph consists of at least 95% of the particular polymorphic form, in some instances the polymorph consists of at least 98% of the particular polymorphic form, preferably at least 98.5%, more preferably at least 99%, yet more preferably at least 99.5%. In some instances the polymorph consists of 100% of the particular polymorphic form i.e. there is no other polymorphic form present.

To the cooled reaction mixture, sodium hydroxide solution may be added over a specific period of time. Sodium hydroxide solution may be made by adding or dissolving sodium hydroxide in suitable solvent. The suitable solvent may be selected from the group consisting of water, C₁₋₄ alcohols or mixture thereof. The solvent, C₁₋₄ alcohol, may be selected from methanol, ethanol, isopropanol, n-butanol, isobutanol.

The amount of sodium hydroxide used may range from 1.1 to 2.1 moles with respect to starting material that is dolutegravir base or hydroxyprotected dolutegravir.

The addition of sodium hydroxide solution to dolutegravir solution or vice versa may be carried out in less than 60 minutes, preferably between 30 and 60 minutes. The time period may be around 30 minutes or less than 30 minutes. This time of addition is also very crucial for formation of polymorph because an increase in this time of addition may result in the formation of a different polymorph. Suitably, the sodium hydroxide solution may be added to the dolutegravir solution, or the dolutegravir solution may be added to the sodium hydroxide solution.

The reaction mass may be maintained at a temperature—ranging from −10° to 20° C., preferably ranging from −10° to 15° C. and for a specific period of time.

The reaction mass may be maintained for 30 minutes to 3 hours.

Finally the reaction mass may be filtered and the product obtained dried.

Maintaining the reaction mixture at the lower temperature is also important for formation of the desired polymorph.

Alternatively polymorph 1B may directly be obtained from hydroxy protected dolutegravir wherein hydroxy protected dolutegravir is deprotected using processes known in the art to get dolutegravir base. Suitably, dolutegravir base may be isolated before being converted to polymorph D, wherein isolated means that solid dolutegravir base is obtained from the deprotection reaction mass before being used in the process for preparing polymorph 1B (i.e. in step a) in the claimed process) and a suitable solvent may be added to it. Suitably, dolutegravir base may be converted to polymorph 1B in situ, wherein in situ means that the dolutegravir base is present in a solution and is not isolated before being used in the process for preparing polymorph 1B (i.e. in step a) in the claimed process), and a suitable solvent may be added to it.

The suitable solvent may be selected from the group consisting of C₁₋₄ alcohol, halogenated hydrocarbons or mixtures thereof. The solvent may be selected from methanol, ethanol, isopropanol, n-butanol, isobutanol, methylene dichloride (MDC), toluene, preferably ethanol.

This reaction mixture may be cooled to a temperature—ranging from −10° to 20° C., preferably ranging from −10° to 15° C.

Cooling at this temperature range is important to get the desired polymorph Form 1B in pure polymorphic form.

As noted above, pure polymorphic form means a polymorph which is substantially free of any other polymorphic form, wherein “substantially free of any other polymorphic form” means the polymorph consists of at least 95% of the particular polymorphic form, in some instances the polymorph consists of at least 98% of the particular polymorphic form, preferably at least 98.5%, more preferably at least 99%, yet more preferably at least 99.5%. In some instances the polymorph consists of 100% of the particular polymorphic form i.e. there is no other polymorphic form present.

To the cooled reaction mixture, sodium hydroxide solution may be added over specific period of time. Sodium hydroxide solution may be made by adding or dissolving sodium hydroxide in a suitable solvent. The suitable solvent may be selected from the group consisting of water, C₁₋₄ alcohols or mixture thereof. The solvent, C₁₋₄ alcohol, may be selected from methanol, ethanol, isopropanol, n-butanol, isobutanol.

The amount of sodium hydroxide used may range from 1.1 to 2.1 moles with respect to starting material that is dolutegravir base or hydroxy protected dolutegravir.

The addition of sodium hydroxide solution to dolutegravir solution or vice versa, may be carried out in less than 60 minutes, preferably between 30 and 60 minutes. The time period may be around 30 minutes or less than 30 minutes. This time of addition is also very crucial for formation of polymorph because an increase in this time of addition may result in the formation of a different polymorph. Suitably, the sodium hydroxide solution may be added to the dolutegravir solution, or the dolutegravir solution may be added to the sodium hydroxide solution.

The reaction mass may be maintained at a temperature ranging from −10° to 20° C., preferably ranging from −10° to 15° C. and for a specific period of time.

The reaction mass may be maintained for 30 minutes to 3 hours.

Finally the reaction mass may be filtered and the product obtained dried.

Maintaining the reaction mixture at the lower temperature is also important for formation of the desired polymorph.

Any other known crystalline form of dolutegravir sodium may also be converted into novel Form 1A or Form 1B via dolutegravir base. Suitably, dolutegravir base may be isolated before being converted to novel Form 1A or Form 1B, wherein isolated means that solid dolutegravir base is obtained from the deprotection reaction mass before being used in the process for preparing novel Form 1A or Form 1B (i.e. in step a) in the claimed process). Suitably, dolutegravir base may be converted to novel Form 1A or Form 1B in situ, wherein in situ means that the dolutegravir base is present in a solution and is not isolated during the process before being used in the process for preparing Form 1A or Form 1B (i.e. in step a) in the respective claimed processes).

To convert known crystalline form to polymorph 1A, first crystalline dolutegravir sodium may be dissolved in a suitable solvent. The suitable solvent used may be selected from the group consisting of water. C₁₋₄ alcohols, halogenated hydrocarbons or mixtures thereof. The solvent may be selected from water, methanol, ethanol, isopropanol, n-butanol, isobutanol, methylene dichloride (MDC), toluene, or mixtures thereof.

The suitable temperature of mixing in the first step ranges from 20° to 35° C., preferably ranging from 25° to 30° C.

After addition of the solvent, suitable acid may be added to the reaction mass. The acid may be inorganic or organic acid selected from hydrochloric acid, sulphuric acid, acetic acid, fumaric acid, citric acid and the like.

The obtained dolutegravir base may be extracted in suitable solvent.

Suitable solvent is C₁₋₄ alcohols, halogenated hydrocarbons or mixtures thereof.

The solvent may be selected from methanol, ethanol, isopropanol, n-butanol, isobutanol, methylene dichloride (MDC), toluene, or mixtures thereof.

Dolutegravir base obtained is present in the organic layer collected after extraction. This layer may then be mixed with solvent which is selected from methanol, ethanol, isopropanol, n-butanol, isobutanol.

This reaction mixture may be cooled to a temperature ranging from −10° to 20° C., preferably ranging from −10° to 15° C.

The reaction works well at this temperature range and provides pure polymorphic form 1A.

As noted above, pure polymorphic form means a polymorph which is substantially free of any other polymorphic form, wherein “substantially free of any other polymorphic form” means the polymorph consists of at least 95% of the particular polymorphic form, in some instances the polymorph consists of at least 98% of the particular polymorphic form, preferably at least 98.5%, more preferably at least 99%, yet more preferably at least 99.5%. In some instances the polymorph consists of 100% of the particular polymorphic form i.e. there is no other polymorphic form present.

To the cooled reaction mixture, sodium hydroxide solution may be added over specific period of time. Sodium hydroxide solution may be made by adding or dissolving sodium hydroxide in a suitable solvent. The suitable solvent used may be selected from the group consisting of water, C₁₋₄ alcohols or mixture thereof. The solvent, C₁₋₄ alcohols, may be selected methanol, ethanol, isopropanol, n-butanol, isobutanol.

The amount of sodium hydroxide used may range from 1.1 to 2.1 moles with respect to starting material that is dolutegravir sodium salt.

The addition of sodium hydroxide solution to dolutegravir solution or vice versa may be carried out in less than 60 minutes, preferably between 30 and 60 minutes. The time period may be around 30 minutes or less than 30 minutes. This time of addition is also very crucial for formation of polymorph because an increase in this time of addition may result in the formation of a mixture of polymorphs or the formation of another polymorph. Suitably, the sodium hydroxide solution may be added to the dolutegravir solution, or the dolutegravir solution may be added to the sodium hydroxide solution.

The reaction mass may be maintained at a temperature ranging from −10° to 20° C., preferably ranging from −10° to 15° C. and for a specific period of time.

The reaction mass may be maintained for 30 minutes to 3 hours.

Maintaining the reaction mixture at the lower temperature is also important for formation of the desired polymorph.

Finally the reaction mass may be filtered and the product obtained dried.

Crystalline dolutegravir sodium may be converted to form 1B by dissolving crystalline dolutegravir sodium in a suitable solvent. The suitable solvent used may be selected from the group consisting of water, C₁₋₄ alcohols, halogenated hydrocarbons or mixtures thereof. The solvent may be selected from water, methanol, ethanol, isopropanol, n-butanol, isobutanol, methylene dichloride (MDC), toluene, or mixtures thereof.

The suitable temperature of mixing in the first step ranges from 20° to 35° C., preferably ranging from 25° to 30° C.

After addition of the solvent above, suitable acid may be added to the reaction mass. The acid may be inorganic or organic acid selected from hydrochloric acid, sulphuric acid, acetic acid, fumaric acid, citric acid and the like.

The obtained dolutegravir base may be extracted in suitable solvent.

Suitable solvent is C₁₋₄ alcohols, halogenated hydrocarbons or mixtures thereof.

The solvent may be selected from methanol, ethanol, isopropanol, n-butanol, isobutanol, methylene dichloride (MDC), toluene, or mixtures thereof.

The organic layer containing dolutegravir base may be mixed with the solvent which is selected from methanol, ethanol, isopropanol, n-butanol, isobutanol.

This reaction mixture may be cooled to a temperature ranging from −10° to 20° C., preferably ranging from −10° to 15° C. This temperature range is important to get the pure polymorphic form 1B.

As noted above, pure polymorphic form means a polymorph which is substantially free of any other polymorphic form, wherein “substantially free of any other polymorphic form” means the polymorph consists of at least 95% of the particular polymorphic form, in some instances the polymorph consists of at least 98% of the particular polymorphic form, preferably at least 98.5%, more preferably at least 99%, yet more preferably at least 99.5%. In some instances the polymorph consists of 100% of the particular polymorphic form i.e. there is no other polymorphic form present.

To the cooled reaction mixture, sodium hydroxide solution may be added over specific period of time. Sodium hydroxide solution may be made by adding or dissolving sodium hydroxide in a suitable solvent. The suitable solvent may be selected from the group consisting of water, C₁₋₄ alcohols or mixture thereof. The solvent, C₁₋₄ alcohol, may be selected from methanol, ethanol, isopropanol, n-butanol, isobutanol.

The amount of sodium hydroxide used may range from 1.1 to 2.1 moles with respect to starting material that is dolutegravir sodium salt.

The addition of sodium hydroxide solution to dolutegravir solution or vice versa may be carried out in less than 60 minutes, preferably between 30 and 60 minutes. The time period may be around 30 minutes or less than 30 minutes. This time of addition is also very crucial for formation of polymorph because an increase in this time of addition may result in the formation of a different polymorph. Suitably, the sodium hydroxide solution may be added to the dolutegravir solution, or the dolutegravir solution may be added to the sodium hydroxide solution.

The reaction mass may be maintained at a temperature ranging from −10° to 20° C., preferably ranging from −10° to 15° C. and for a specific period of time.

The reaction mass may be maintained for 30 minutes to 3 hours.

Finally the reaction mass may be filtered and the product obtained dried.

Maintaining the reaction mixture at the lower temperature is also important for formation of the desired polymorph.

The compound of formula (I) in crystalline form 1A or form 1B may be isolated from the reaction mixture using techniques well known in the art such as, decanting, filtration, centrifugation and the like.

Further, the so obtained compound may be dried using well known processes.

As discussed previously, WO2010068253 and U.S. Pat. No. 9,242,986 disclose a polymorphic form of dolutegravir sodium and a polymorphic form of dolutegravir sodium hydrate. Comparative intrinsic dissolution, bulk density and tapped density may be studied with either of these polymorphs or other polymorphs and the polymorphs of the present invention.

The following data illustrates the dissolution of the crystalline form of dolutegravir sodium disclosed in WO2010068253 (i.e. not the hydrate), compared to crystalline Form 1A of the present invention (i.e. crystalline Form 1A of dolutegravir sodium). This data is graphically represented in FIG. 5.

| | % Release of | |
|---|---|---|
| | dolutegravir | % Release of |
| | sodium Form | crystalline form |
| | (crystalline form | 1A of dolutegravir |
| | of dolutegravir | sodium (Form 1A of |
| | sodium disclosed | dolutegravir sodium of |
| Time in minutes | in WO2010068253) | the present invention) |
| 5 | 5 | 26 |
| 15 | 13 | 45 |
| 30 | 20 | 73 |
| 60 | 56 | 66 |
| 120 | 68 | 79 |
| 240 | 74 | 82 |
| 360 | 72 | 81 |
| 480 | 73 | 85 |
| 600 | 73 | 86 |
| 720 | 74 | 82 |

Experimental Conditions:

Dissolution medium: 0.01M pH 6.8 phosphate buffer containing 0.25% w/v sodium dodecyl sulfate (SDS)

Temperature: 37° C.

Rotation speed: 50 rpm

The above data indicates that intrinsic dissolution of both polymorphs are comparable.

Suitable methods for determining the bulk density and tapped density of the crystalline form 1A of dolutegravir sodium of the present invention and the crystalline form of dolutegravir sodium form disclosed in WO2010068253 will be well known to the skilled chemist. For example, USP <616> method I may be used.

The crystalline Form 1A of dolutegravir sodium of the present invention preferably exhibits bulk density in the range of 0.4-0.5 g/ml and tapped density is in the range of 0.7-0.8 g/ml. In comparison the crystalline form of dolutegravir sodium disclosed in WO2010068253 exhibits bulk density in the range of 0.2-0.3 g/ml and tapped density in the range of 0.4-0.5 g/ml.

These higher densities of crystalline Form 1A dolutegravir sodium are advantageous in formulation specifically in tablet formulation. For example, it gives better compressibility. As such, crystalline Form 1A of the present invention is able to provide comparable dissolution characteristics but with the advantage of the Form being more suitable for formulation.

Accordingly, it is preferred that the crystalline Form 1A of the present invention is characterised by a bulk density in the range of 0.4-0.5 g/ml. Further it is preferred that the Form 1A of the present invention is characterised by a tapped density is in the range of 0.7-0.8 g/ml.

Dolutegravir base and dolutegravir sodium salt may be made by the processes known in the art before crystallization or recrystallization.

Yet another aspect of present invention provides pharmaceutical composition comprising new polymorphic forms 1A or 1B of dolutegravir sodium in combination with a pharmaceutically acceptable carrier. In addition to active ingredient(s) the pharmaceutical composition of the present invention may contain one or more pharmaceutically acceptable excipients/ingredients.

In another aspect of present invention provides pharmaceutical composition comprising effective amount of new polymorphic forms 1A or 1B of dolutegravir sodium with one or more therapeutic agents in combination with a pharmaceutically acceptable excipients/ingredients.

The therapeutic agents means drugs which are active against HIV infection, AIDS, and AIDS related conditions. Therapeutic agents selected from the group consisting of nucleotide reverse transcriptase inhibitors, nucleoside reverse transcriptase inhibitors (NRTIs), non-nucleoside reverse transcriptase inhibitors (NNRTIs), protease inhibitors, CCR5 antagonists, CXCR4 antagonists, fusion inhibitors, maturation inhibitors, and integrase inhibitors.

The composition of the present invention may be formulated into variety of dosage forms, such as tablets, capsules, pills, caplets, lozenges, dispersible granules, dry powder syrup, ready to use suspension; parenteral dosage forms available in the art; various inhalation formulations; transdermal formulations, and the like. These formulations may be prepared using processes known in the art.

Still another aspect on the invention relates to a method of treating HIV infection and related conditions in a patient, said method comprising administering to the patient a pharmaceutical formulation comprising therapeutically effective amount of a new polymorphic forms 1A or 1B of dolutegravir sodium.

### BRIEF DESCRIPTION OF ACCOMPANYING DRAWINGS

FIG. 1 shows the powder X-ray diffraction pattern of solid crystalline Form 1A of dolutegravir sodium.

FIG. 2 shows the powder X-ray diffraction pattern of solid crystalline Form 1B of dolutegravir sodium.

FIG. 3 shows the powder infrared spectrum of solid crystalline Form 1A of dolutegravir sodium.

FIG. 4 shows the powder infrared spectrum of solid crystalline Form 1B of dolutegravir sodium.

FIG. 5 is a graphical comparison of the intrinsic dissolution of the solid crystalline Form 1A of dolutegravir sodium of the present invention and the crystalline form of dolutegravir sodium disclosed in WO2010068253. The solid line represents the dissolution of solid crystalline Form 1A of dolutegravir sodium of the present invention, with the dotted line representing the dissolution of the crystalline form of dolutegravir sodium disclosed in WO2010068253.

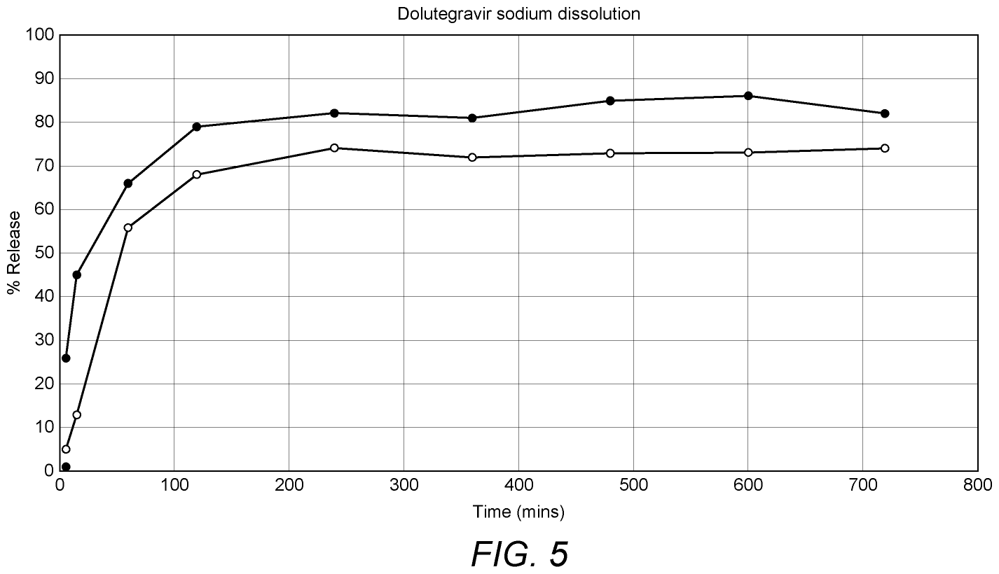

The present invention relating to new polymorphs of dolutegravir sodium, processes for the preparation of said polymorphs, and pharmaceutical compositions containing said polymorphs may also be described with reference to the following statements, which are not to be taken to be claims, but may form the basis for subsequent claims.

Statement 1: A crystalline polymorph 1A of dolutegravir sodium having an X-ray powder diffraction pattern comprising peaks, in terms of degrees 2-theta, at 15.66°, 20.29° and 22.51°±0.2° 2 theta.

Statement 2: A crystalline polymorph 1A of dolutegravir sodium according to statement 1 having an X-ray powder diffraction pattern comprising further peaks, in terms of degrees 2-theta, at 8.00° and 25.53°±0.2° 2 theta.

Statement 3: A crystalline polymorph 1A of dolutegravir sodium according to statement 1 or 2 having an X-ray powder diffraction pattern comprising further peaks, in terms of degrees 2-theta, at 18.36°, 21.11°, and 23.49°±0.2° 2 theta.

Statement 4: A crystalline polymorph 1A of dolutegravir sodium according to any one of statements 1 to 3 having an X-ray powder diffraction pattern comprising further peaks, in terms of degrees 2-theta, at 8.76°, 9.68°, 10.95°, 11.94°, 12.74°, 13.15°, 17.00°, 19.84°, 26.72° and 27.48°±0.2° 2 theta.

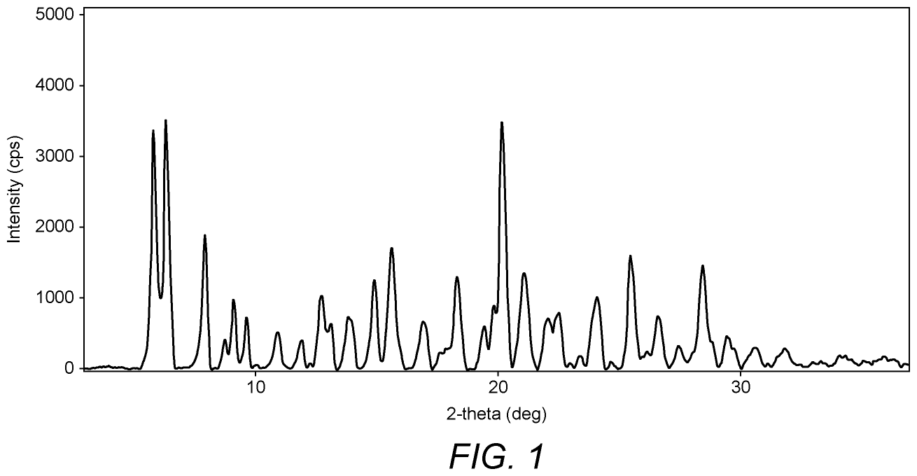

Statement 5: A crystalline polymorph 1A of dolutegravir sodium according to any preceding statement characterised by an X-ray powder diffraction pattern substantially as depicted in FIG. 1.

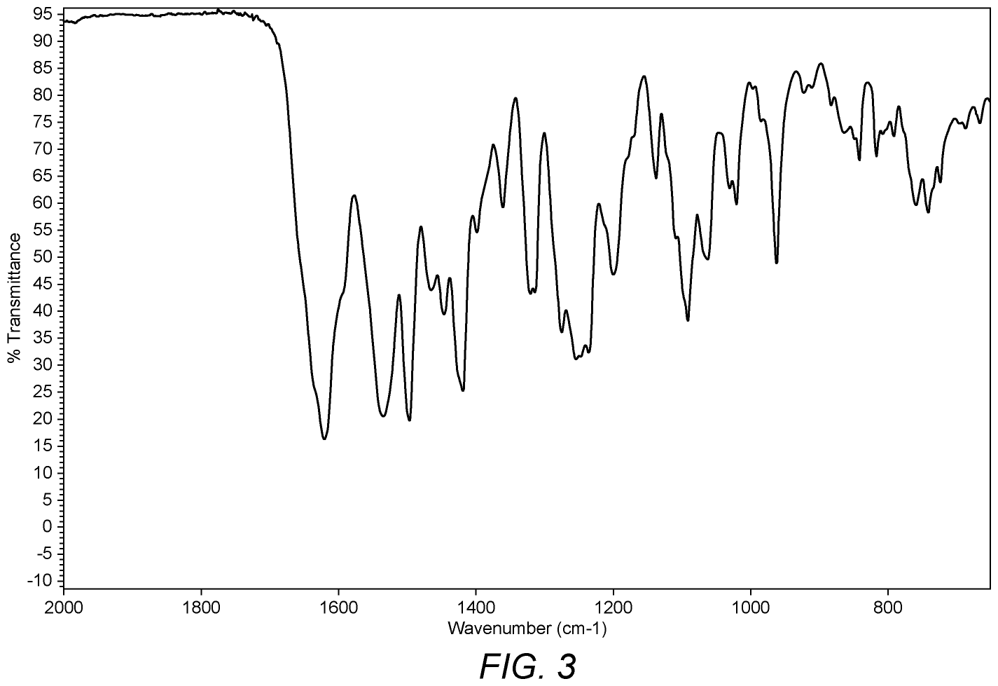

Statement 6: A crystalline polymorph 1A of dolutegravir sodium according to any preceding statement characterised by an infrared spectrum substantially as depicted in FIG. 3.

Statement 7: A crystalline polymorph 1A of dolutegravir sodium according to any preceding statement characterised by a bulk density ranging from 0.3-0.6 g/ml.

Statement 8: A crystalline polymorph 1A of dolutegravir sodium according to statement 7 characterised by a bulk density ranging from 0.4-0.5 g/ml.

Statement 9: A crystalline polymorph 1A of dolutegravir sodium according to any preceding statement characterised by a tapped density ranging from 0.6-0.9 g/ml.

Statement 10: A crystalline polymorph 1A of dolutegravir sodium according to statement 7 characterised by a tapped density ranging from 0.7-0.8 g/ml.

Statement 11: A crystalline polymorph 1A of dolutegravir sodium characterised by an X-ray powder diffraction pattern substantially as depicted in FIG. 1.

Statement 12: A crystalline polymorph 1A of dolutegravir sodium characterised by an infrared spectrum substantially as depicted in FIG. 3.

Statement 13: A crystalline polymorph 1A of dolutegravir sodium characterised by a bulk density ranging from 0.3-0.6 g/ml.

Statement 14: A process for preparing crystalline polymorph 1A of dolutegravir sodium as defined in any preceding statement, the process comprising the steps of:
a) preparing a mixture of dolutegravir base with methanol,
b) cooling the reaction mixture from step a) to a temperature ranging from −10° C. to 20° C.
c) preparing a solution of sodium hydroxide in a solvent,
d) mixing the solutions from steps b) and c) over a period of time less than 60 minutes at a temperature ranging from −10° C. to 20° C.
e) maintaining the reaction mass from step d) at a temperature in the range of −10° C. to 20° C. over a period of time ranging from 30 minutes to 3 hours, and
f) isolating crystalline polymorph 1A of dolutegravir sodium.

Statement 15: A process according to statement 14 wherein the dolutegravir base used in step a) is obtained by any known process.

Statement 16: A process according to statement 14 or 15 wherein the dolutegravir base used in step a) is obtained in situ by deprotecting hydroxy protected dolutegravir.

Statement 17: A process according to statement 14 or 15 wherein the dolutegravir base used in step a) is obtained by deprotecting hydroxy protected dolutegravir and used after isolation from the reaction mass.

Statement 18: A process according to statement 14 wherein the dolutegravir base used in step a) is obtained from crystalline dolutegravir sodium.

Statement 19: A process according to any one of statements 14 to 18, wherein the temperature in step a) ranges from 20° C. to 35° C.

Statement 20: A process according to statement 19, wherein the temperature in step a) ranges from 25° C. to 30° C.

Statement 21: A process according to any one of statements 14 to 20 wherein the temperature in step b) ranges from −10° C. to 10° C.

Statement 22: A process according to any one of statements 14 to 21 wherein the temperature in step b) ranges from −7° C. to −5° C.

Statement 23: A process according to any one of statements 14 to 21 wherein the temperature in step b) ranges from −5° C. to 10° C.

Statement 24: A process according to statement 23 wherein the temperature in step b) ranges from −5° C. to 0° C.

Statement 25: A process according to statement 23 wherein the temperature in step b) ranges from 0° C. to 5° C.

Statement 26: A process according to statement 23 wherein the temperature in step b) ranges from 5° C. to 10° C.

Statement 27: A process according to any one of statements 14 to 26 wherein the solvent in step c) is selected from the group consisting of water, methanol, ethanol, isopropanol, n-butanol and isobutanol.

Statement 28: A process according to statement 27 wherein the solvent in step c) is water or methanol.

Statement 29: A process according to any one of statements 14 to 28 wherein the period of time in step d) is less than 30 minutes.

Statement 30: A process according to any one of statements 14 to 28 wherein the period of time in step d) ranges from 30 minutes to 60 minutes.

Statement 31: A process according to any one of statements 14 to 30 wherein the amount of sodium hydroxide used ranges from 1.1 to 2.1 moles with respect to starting material that is dolutegravir base or dolutegravir sodium or hydroxyprotected dolutegravir.

Statement 32: A process according to any one of statements 14 to 31 wherein the solution from step c) is added to the solution from step b) over a period of time ranging from 30 to 60 minutes at a temperature ranging from −10° C. to 20° C.

Statement 33: A process according to statement 32 wherein, in step d), the solution from step c) is added to the solution from step b) over a period of time ranging from 25 to 35 minutes.

Statement 34: A process according to any one of statements 14 to 31 wherein, in step d), the solution from step b) is added to the solution from step c) over a period of time ranging from 30 to 60 minutes at a temperature ranging from −10° C. to 20° C.

Statement 35: A process according to statement 34 wherein, in step d), the solution from step b) is added to the solution from step c) over a period of time ranging from 30 to 45 minutes.

Statement 36: A process according to any one of statements 14 to 35 wherein the temperature in step d) ranges from −10° C. to 10° C.

Statement 37: A process according to statement 36 wherein the temperature in step d) ranges from −5° C. to −3° C.

Statement 38: A process according to statement 36 wherein the temperature in step d) ranges from −5° C. to 10° C.

Statement 39: A process according to statement 38 wherein the temperature in step d) ranges from 0° C. to 10° C.

Statement 40: A process according to any one of statements 14 to 39 wherein the temperature in step e) ranges from −10° C. to 10° C.

Statement 41: A process according to statement 40 wherein the temperature in step e) ranges from −4° C. to −2° C.

Statement 42: A process according to statement 40 wherein the temperature in step e) ranges from −5° C. to 10° C.

Statement 43: A process according to statement 42 wherein the temperature in step e) ranges from 0° C. to 10° C.

Statement 44: A process according to any one of statements 14 to 43, wherein step f) comprises filtering the reaction mass from step e) and drying the filtration residue under vacuum at a temperature ranging from 45° C. to 85° C., preferably ranging from 50° C. to 60° C., more preferably at 55° C.

Statement 45: A process according to statement 44, wherein step further comprises washing the filtration residue with methanol before drying.

Statement 46: A process according to statement 14, wherein the temperature in step a) ranges from 25° C. to 30° C., the temperature in step b) ranges from 0° C. to 5° C., wherein the solvent in step c) is water, wherein the solution from step c) is added to the solution from step b) in step d), wherein the period of time in step d) is less than 30 minutes, wherein the temperature in step d) ranges from 0° C. to 5° C., wherein the temperature in step e) ranges from 0° C. to 5° C., wherein the period of time in step e) is 1 hour or less, and wherein step f) comprises filtering the reaction mass from step e), washing the filtration residue with methanol and drying the filtration residue under vacuum at a temperature of 55° C.

Statement 47: A process according to statement 14, wherein the temperature in step a) ranges from 25° C. to 30° C., the temperature in step b) ranges from 0° C. to 5° C., wherein the solvent in step c) is methanol, wherein the solution from step c) is added to the solution from step b) in step d), wherein the period of time in step d) is less than 30 minutes, wherein the temperature in step d) ranges from 0° C. to 5° C., wherein the temperature in step e) ranges from 0° C. to 5° C., wherein the period of time in step e) is 1 hour or less, and wherein step f) comprises filtering the reaction mass from step e), washing the filtration residue with methanol and drying the filtration residue under vacuum at a temperature of 55° C.

Statement 48: A process according to statement 14, wherein the temperature in step a) ranges from 25° C. to 30° C., the temperature in step b) ranges from 5° C. to 10° C., wherein the solvent in step c) is water, wherein the solution from step c) is added to the solution from step b) in step d), wherein the period of time in step d) is less than 30 minutes, wherein the temperature in step d) ranges from 0° C. to 5° C., wherein the temperature in step e) ranges from 0° C. to 5° C., wherein the period of time in step e) is 1 hour or less, and wherein step f) comprises filtering the reaction mass from step e), washing the filtration residue with methanol and drying the filtration residue under vacuum at a temperature of 55° C.

Statement 49: A process according to statement 14, wherein the temperature in step a) ranges from 25° C. to 30° C., the temperature in step b) ranges from −5° C. to 0° C., wherein the solvent in step c) is water, wherein the solution from step c) is added to the solution from step b) in step d), wherein the period of time in step d) is less than 30 minutes, wherein the temperature in step d) ranges from 5° C. to 10° C., wherein the temperature in step e) ranges from 5° C. to 10° C., wherein the period of time in step e) is 1 hour or less, and wherein step f) comprises filtering the reaction mass from step e), washing the filtration residue with methanol and drying the filtration residue under vacuum at a temperature of 55° C.

Statement 50: A process according to statement 14, wherein the dolutegravir base used in step a) is obtained in situ by deprotecting hydroxy protected dolutegravir, the temperature in step b) ranges from 0° C. to 5° C., wherein the solvent in step c) is water, wherein the solution from step c) is added to the solution from step b) in step d), wherein the period of time in step d) is less than 30 minutes, wherein the temperature in step d) ranges from 0° C. to 5° C., wherein the temperature in step e) ranges from 0° C. to 5° C., wherein the period of time in step e) is 1 hour or less, and wherein step f) comprises filtering the reaction mass from step e), washing the filtration residue with methanol and drying the filtration residue under vacuum at a temperature of 55° C.

Statement 51: A process according to statement 14, wherein the dolutegravir base used in step a) is obtained from crystalline dolutegravir sodium, the temperature in step b) ranges from 0° C. to 5° C., wherein the solvent in step c) is water, wherein the solution from step c) is added to the solution from step b) in step d), wherein the period of time in step d) is less than 30 minutes, wherein the temperature in step d) ranges from 0° C. to 5° C., wherein the temperature in step e) ranges from 0° C. to 5° C., wherein the period of time in step e) is 1 hour or less, and wherein step f) comprises filtering the reaction mass from step e), washing the filtration residue with methanol and drying the filtration residue under vacuum at a temperature of 55° C.

Statement 52: A process according to statement 14, wherein the temperature in step a) ranges from 25° C. to 30° C., the temperature in step b) ranges from −7° C. to −5° C. wherein the solvent in step c) is water, wherein the solution from step b) is added to the solution from step c) in step d), wherein the period of time in step d) ranges from 30 minutes to 45 minutes, wherein the temperature in step d) ranges from −5° C. to −3° C., wherein the temperature in step e) ranges from −4° C. to −2° C., wherein the period of time in step e) is 1 hour or less, and wherein step f) comprises filtering the reaction mass from step e) and drying the filtration residue at a temperature of 80° C.

Statement 53: A process according to any one of statements 14 to 52, wherein the dolutegravir base used in step a) is formed in situ by a process that comprises the steps of:
1) dissolving crystalline dolutegravir sodium in a solvent, at a temperature ranging from 20° C. to 35° C.,
2) adding an acid to the reaction mass of step 1), and
3) extracting dolutegravir base in a solvent.

Statement 54: A process according to statement 53 wherein the temperature in step 1) ranges from 25° C. to 30° C.

Statement 55: A process according to statement 53 or 54 wherein the solvent in step 1) is selected from the group consisting of water, methanol, ethanol, isopropanol, n-butanol, isobutanol, methylene dichloride (MDC), toluene, or mixtures thereof, preferably water.

Statement 56: A process according to any one of statements 53 to 55 wherein the acid is selected from hydrochloric acid, sulphuric acid, acetic acid, fumaric acid and citric acid.

Statement 57: A process according to statement 56 wherein the acid is acetic acid.

Statement 58: A process according to any one of statements 53 to 57 wherein the solvent for extraction in step 3) is selected from methanol, ethanol, isopropanol, n-butanol, isobutanol, methylene dichloride (MDC), toluene or mixtures thereof.

Statement 59: A process according to statement 57 wherein the solvent for extraction in step 3) is methylene dichloride.

Statement 60: A crystalline polymorph 1B of dolutegravir sodium having an X-ray powder diffraction pattern comprising peaks, in terms of degrees 2-theta, at 7.08°, 10.82°, 12.95°, 15.93° and 18.59°±0.2° 2 theta.

Statement 61: A crystalline polymorph 1B of dolutegravir sodium according to statement 60 having an X-ray powder diffraction pattern comprising peaks, in terms of degrees 2-theta, at 13.82°, 17.44°, 20.09°, 22.78°, 25.41°, 26.35°, 28.81°±0.2° 2 theta.

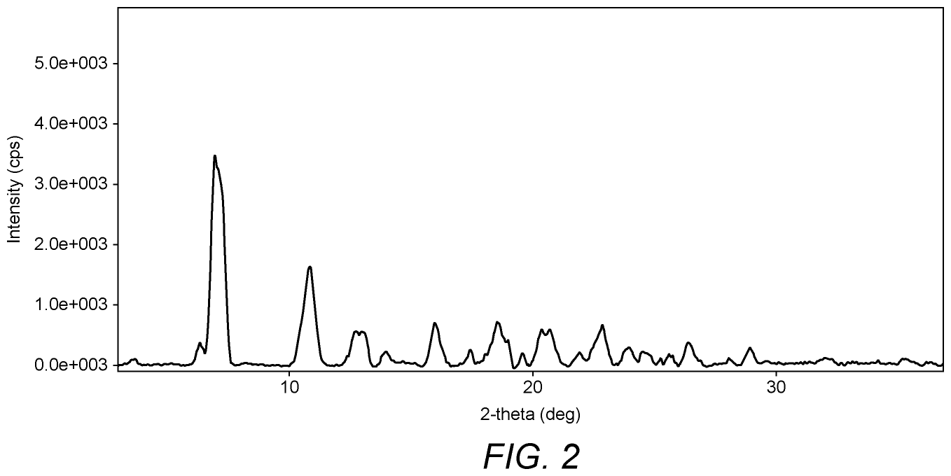

Statement 62: A crystalline polymorph 1B of dolutegravir sodium according to statement 60 or 61 characterised by an X-ray powder diffraction pattern substantially as depicted in FIG. 2.

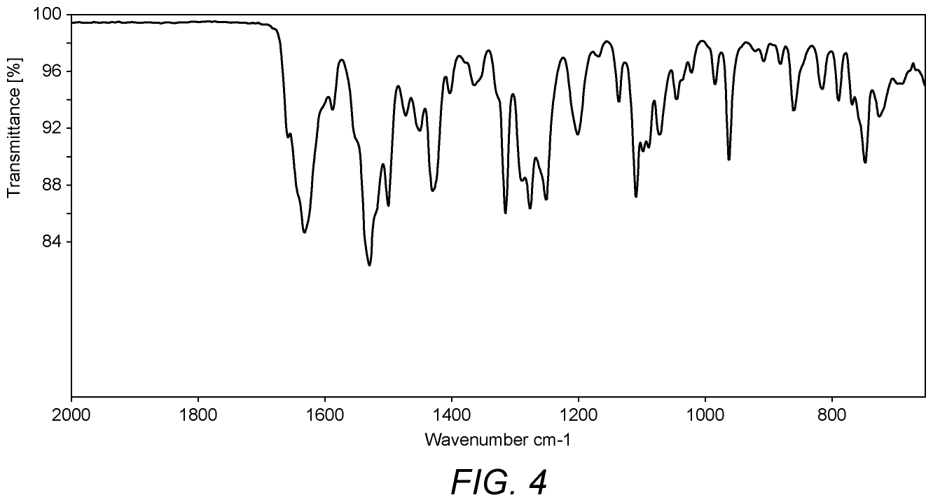

Statement 63: A crystalline polymorph 1B of dolutegravir sodium according to statement 60, 61, or 62 characterised by an infrared spectrum substantially as depicted in FIG. 4.

Statement 64: A crystalline polymorph 1B of dolutegravir sodium characterised by an X-ray powder diffraction pattern substantially as depicted in FIG. 2.

Statement 65: A crystalline polymorph 1B of dolutegravir sodium characterised by an infrared spectrum substantially as depicted in FIG. 4.

Statement 66: A process for preparing crystalline polymorph 1B of dolutegravir sodium as defined in any one of statements 60 to 65, the process comprising the steps of:
a) preparing a mixture of dolutegravir base with ethanol,
b) cooling the reaction mixture from step a) to a temperature ranging from −10° C. to 20° C.,
c) preparing a solution of sodium hydroxide in a solvent,
d) mixing the solutions from steps b) and c) over a period of time ranging from 30 to 60 minutes at a temperature ranging from −10° C. to 20° C.,
e) maintaining the reaction mass from step d) at a temperature in the range of −10° C. to 20° C. over a period of time ranging from 30 minutes to 3 hours, and
f) isolating crystalline polymorph 1B of dolutegravir sodium.

Statement 67: A process according to statement 66 wherein the dolutegravir base used in step a) is obtained by any know n process.

Statement 68: A process according to statement 66 or 67 wherein the dolutegravir base used in step a) is obtained in situ by deprotecting hydroxy protected dolutegravir.

Statement 69: A process according to statement 66 or 67 wherein the dolutegravir base used in step a) is obtained by deprotecting hydroxy protected dolutegravir and used after isolation from the reaction mass.

Statement 70: A process according to statement 66 wherein the dolutegravir base used in step a) is obtained from crystalline dolutegravir sodium.

Statement 71: A process according to any one of statements 66 to 70 wherein the temperature in step a) ranges from 20° C. to 35° C.

Statement 72: A process according to statement 71 wherein the temperature in step a) ranges from 25° C. to 30° C.

Statement 73: A process according to any one of statements 66 to 72 wherein the solvent in step c) is selected from the group consisting of water, methanol, ethanol, isopropanol, n-butanol and isobutanol.

Statement 74: A process according to statement 73 wherein the solvent in step c) is water or ethanol.

Statement 75: A process according to any one of statements 66 to 74 wherein the amount of sodium hydroxide used ranges from 1.1 to 2.1 moles with respect to the starting material that is dolutegravir base or dolutegravir sodium or hydroxyprotected dolutegravir.

Statement 76: A process according to any one of statements 66 to 75, wherein the dolutegravir base used in step a) is formed in situ by a process that comprises the steps of:
1) dissolving crystalline dolutegravir sodium in a solvent, at a temperature ranging from 20° C. to 35° C.,
2) adding an acid to the reaction mass of step 1), and
3) extracting dolutegravir base in a solvent.

Statement 77: A process according to statement 76 wherein the temperature in step 1) ranges from 25° C. to 30° C.

Statement 78: A process according to statement 76 or 77 wherein the solvent in step 1) is selected from the group consisting of water, methanol, ethanol, isopropanol, n-butanol, isobutanol, methylene dichloride (MDC), toluene, or mixtures thereof.

Statement 79: A process according to statement 78 wherein the solvent in step 1) is water.

Statement 80: A process according to any one of statements 76 to 79 wherein the acid in step 2) is selected from hydrochloric acid, sulphuric acid, acetic acid, fumaric acid and citric acid.

Statement 81: A process according to statement 80 wherein the acid in step 2) is acetic acid.

Statement 82: A process according to any one of statements 76 to 81 wherein the solvent for extraction in step 3) is selected from methanol, ethanol, isopropanol, n-butanol, isobutanol, methylene dichloride (MDC), toluene or mixtures thereof.

Statement 83: A process according to statement 82 wherein the solvent for extraction in step 3) is methylene dichloride.

Statement 84: A process according to any one of statements 66 to 83, wherein step f) comprises filtering the reaction mass from step e) and drying the filtration residue under vacuum at a temperature ranging from 45° C. to 85° C., preferably ranging from 50° C. to 60° C., preferably at 55° C.

Statement 85: A process according to statement 84, wherein step further comprises washing the filtration residue with ethanol before drying.

Statement 86: Crystalline form 1A of dolutegravir sodium prepared according to any one of statements 14 to 59.

Statement 87: Crystalline form 1B of dolutegravir sodium prepared according to any one of statements 66 to 85.

Statement 88: A pharmaceutical composition comprising crystalline Form 1A of dolutegravir sodium as defined in any one of statements 1 to 13 or 86 together with one or more pharmaceutically acceptable excipients.

Statement 89: A pharmaceutical composition comprising crystalline Form 1B of dolutegravir sodium as defined in any one of statements 60 to 65 or 87 together with one or more pharmaceutically acceptable excipients.

Statement 90: A pharmaceutical composition comprising crystalline form 1A of dolutegravir sodium as defined in any one of statements 1 to 13 or 86 in combination with one or more further active pharmaceutical ingredients together with one or more pharmaceutically acceptable excipients.

Statement 91: A pharmaceutical composition comprising crystalline Form 1B of dolutegravir sodium as defined in any one of statements 60 to 65 or 87 in combination with one or more further active pharmaceutical ingredients, together with one or more pharmaceutically acceptable excipients.

Statement 92: A pharmaceutical composition according to any one of statements 88 to 91 wherein the dosage form is selected from a list consisting of tablets, capsules, pills, caplets, lozenges, dispersible granules, dry powder syrup, ready to use suspension, parenteral dosage forms, various inhalation formulations, and transdermal formulations.

Statement 93: A pharmaceutical composition according to statements 92 wherein the dosage form is a tablet.

Statement 94: A pharmaceutical composition according to statement 92 wherein the dosage form is a dry powder syrup.

Statement 95: A pharmaceutical composition according to statement 92 wherein the dosage form is a ready use suspension.

Statement 96: A method of treating HIV infection, AIDS and related conditions in a patient, said method comprising administering to the patient a pharmaceutical formulation comprising a therapeutically effective amount of crystalline Form 1A of dolutegravir sodium as defined in any one of statements 1 to 13 or 86.

Statement 97: A method of treating HIV infection, AIDS and related conditions in a patient, said method comprising administering to the patient a pharmaceutical formulation comprising a therapeutically effective amount of crystalline Form 1B of dolutegravir sodium as defined in any one of statements 60 to 65 or 87.

Statement 98: Crystalline Form 1A of dolutegravir sodium as defined in any one of statements 1 to 13 or 86 for use in treating HIV infection, AIDS and related conditions in a patient.

Statement 99: Crystalline Form 1B of dolutegravir sodium as defined in any one of statements 60 to 65 or 87 for use in treating HIV infection, AIDS and related conditions in a patient.

Statement 100: Use of crystalline form 1A of dolutegravir sodium as defined in any one of statements 1 to 13 or 86 in the manufacture of a medicament for treating HIV infection, AIDS and related conditions in a patient.

Statement 101: Use of crystalline Form 1B of dolutegravir sodium as defined in any one of statements 60 to 65 or 87 in the manufacture of a medicament for treating HIV infection, AIDS and related conditions in a patient.

Statement 102: Crystalline Form 1A of dolutegravir sodium substantially as herein defined with reference to Figure 1 or 3.

Statement 103: Crystalline Form 1B of dolutegravir sodium substantially as herein defined with reference to Figure 2 or 4.

The present invention will now be further illustrated by the following examples, which do not limit the scope of the invention in any way.

### EXAMPLES

### Preparation of Polymorph 1A:

### Example 1

Dolutegravir base 10 g was charged with 100 ml methanol to the reactor at 25-30° C. and cooled to 0-5° C. Aqueous sodium hydroxide (NaOH) (2 g in 11 ml water) was added to reaction mass within 30 minutes at 0-5° C. and maintained for 1 hour at 0-5° C. The reaction mass was filtered and washed with 2 volumes of methanol. The mass obtained was then dried under vacuum at 55° C.

Dry weight—10 g

### Example 2

Dolutegravir base 10 g with 100 ml methanol were charged to the reactor at 25-30° C. and cooled to 0-5° C. Methanolic NaOH (2.5 g in 11 ml methanol) was added to reaction mass within 30 minutes at 0-5° C. and maintained for 1 hour at 0-5° C. The reaction mass was filtered and washed with 2 volumes of methanol. The mass obtained was then dried under vacuum at 55° C.

Dry weight—10 g

### Example 3

Dolutegravir base 150 g with 1.5 litres of methanol were charged into the reactor at 25-30° C. and cooled to 5-10° C. Aqueous NaOH (30 g in 165 ml water) was added to reaction mass within 30 minutes at 0-5° C. and maintained reaction mass for 1 hour at 0-5° C. The reaction mass was filtered and washed with 2 volumes of methanol. The mass obtained was then dried under vacuum at 55° C.

Dry weight—150 g

### Example 4

Dolutegravir base 10 g was charged with 100 ml methanol to the reactor at 25-30° C. and cooled to 0 to −5° C. Aqueous NaOH (2.0 g in 11 ml water) was added to reaction mass within 30 minutes at 5 to 10° C. and maintained for 1 hour at 5 to 10° C. The reaction mass was filtered and washed with 2 volumes of methanol. The mass obtained was then dried under vacuum at 55° C.

Dry weight—10 g

### Example 5

3.0 litres of Tetrahydrofuran (THF) was charged to the four neck round bottom flask at 25-28° C. followed by addition of 300 g of protected hydroxy compound (protected dolutegravir) and 123 g of Lithium bromide anhydrous at 25-28° C. The reaction mass was heated to 60° C. and maintained for 8-10 hours. Alter completion of the reaction, the reaction mass was cooled to 25-28° C. and quenched with 600 ml acetic acid followed by addition of 3.0 litres of methylene dichloride (MDC). 3.0 litres of water was added to the reaction mass and stirred for 10 minutes and the layers were separated. The aqueous layer was extracted with 1.5 litres with MDC and the MDC layer layers were collected. MDC layer was concentrated under vacuum at 40-45° C. and 3 litres of Methanol was added to it. The reaction mass was cooled to 0-5° C. and aqueous NaOH (58 g in 320 ml water) was added to it within 30 minutes. The reaction mass was maintained at 0-5° C. for 1 hour followed by filtration and washing with 2 volumes of methanol. Finally it was dried under vacuum at 55° C.

Dry weight—250 g

### Example 6

12 g of crystalline dolutegravir sodium was charged along with 120 ml water into the reaction flask and stirred at 25-30° C. for 15 minutes. To this mixture, 6 ml Acetic acid was added at once and stirred for 30 minutes. Further, 120 ml methylene dichloride (MDC) was added to above reaction mass and stirred for 30 minutes. The layers were allowed to settle and separated. The organic layer was collected and distilled below 45° C. to obtain Oil. To this oil 100 ml methanol was added at 25-30° C. and cooled to 0-5° C. Aqueous NaOH (2 g in 11 ml water) was added to the reaction mass within 30 minutes at 0-5° C. The reaction mass was maintained at 0-5° C. for 1 hour, filtered and washed with 2 volume methanol. And dried in vacuum oven at 55° C.

Dry weight—10 g

### Example 7

0.2 Kg of sodium hydroxide was dissolved in 1.1 litre purified water along with 5.0 litres of methanol to at 25-30° C. The reaction mass was cooled to −6 to −3° C. To another reactor, 10.0 litres of methanol was charged followed by addition of 1.0 Kg dolutegravir base. Again the reaction mass was cooled to −7 to −5° C. The slurry of dolutegravir base was then added to the solution of sodium hydroxide within 30-45 minutes by maintaining the temperature at −5 to −3° C. and then washed with 1 litre methanol. The slurry was stirred for 1 hour at −4 to −2° C. The reaction mass was filtered and dried in an oven at 80±2° C. for 2 hours.

Dry weight—1.1 Kg

### Preparation of Polymorph 1B:

### Example 8

Dolutegravir base 10 g was charged with 100 ml ethanol to the reactor at 25-30° C. and cooled to 0-5° C. Aqueous NaOH (2 g in 11 ml water) was added to the reaction mass within 30 minutes at 0-5° C. and maintained for 2 hours at 0-5° C. The reaction mass was filtered and washed with 2 volumes of ethanol. T he mass obtained was then dried under vacuum at 55° C.

Dry weight—10 g

### Example 9

Dolutegravir base 10 g with 100 ml ethanol were charged into the reactor at 25-30° C. and cooled to 0-5° C. Ethanolic NaOH solution (2.5 g in 11 ml ethanol) was added to the reaction mass within 30 minutes at 0-5° C. and maintained reaction mass for 1 hour at 0-5° C. The reaction mass was filtered and washed with 2 volumes of ethanol. The mass obtained was then dried under vacuum at 55° C.

Dry weight—10 g

### Example 10

Dolutegravir base 10 g was charged with 100 ml ethanol to the reactor at 25-30° C. and cooled to 5-10° C. Aqueous NaOH (2 g in 11 ml water) was added to the reaction mass within 30 minutes at 5-10° C. and maintained reaction mass for 2 hours at 5-10° C. The reaction mass was filtered and washed with 2 volumes of ethanol. The mass obtained was then dried under vacuum at 55° C.

Dry weight—10 g

### Example 11

Dolutegravir base 10 g was charged with 100 ml ethanol to the reactor at 25-30° C. and cooled reaction mass to 0 to −5° C. Aqueous NaOH (2 g in 11 ml water) was added to the reaction mass at 0 to −5° C. and maintained for 2 hours at 0 to −5° C. The reaction mass filtered and washed with 2 volumes of ethanol. The mass obtained was then dried under vacuum at 55° C.

Dry weight—10 g

### Example 12

3.0 litres of Tetrahydrofuran (THF) was charged to the four neck round bottom flask at 25-28° C. followed by addition of 300 g of protected hydroxy compound and 123 g of Lithium bromide anhydrous. The reaction mass was heated to 60° C. and maintained for 8-10 hours. After completion of the reaction, the reaction mass was cooled to 25-28° C. and quenched with 600 ml acetic acid followed by addition of 3.0 litres of methylene dichloride (MDC). 3.0 litres of water was added to the reaction mass and stirred for 10 minutes and the layers were separated. The aqueous layer was extracted with 1.5 litres with MDC 2 times and the MDC layers were collected. MDC layer was concentrated under vacuum at 40-45° C. and 3 litres of ethanol was added to it. The reaction mass was then cooled to 0-5° C. and aqueous NaOH (58 g in 320 ml water) was added to it within 30 minutes. The reaction mass was maintained at 0-5° C. for 2 hours followed by filtration and washing with 2 volumes of ethanol. Finally it was dried under vacuum at 55° C.

Dry weight—260 g

### Example 13

12 g of crystalline dolutegravir sodium was charged along with 120 ml water into the reaction flask and stirred at 25-30° C. for 15 minutes. To this mixture, 6 ml of Acetic acid was added at once and stirred for 30 minutes. Further, 120 ml methylene dichloride (MDC) was added to above reaction mass and stirred for 30 minutes. The layers were allowed to settle and separated. The organic layer was collected and distilled below 45° C. to obtain Oil. To this oil 100 ml ethanol was added at 25-30° C. and cooled to 0-5° C. Aqueous NaOH (2 g in 11 ml water) was added to the reaction mass within 30 minutes at 0-5° C. The reaction mass was maintained at 0-5° C. for 2 hours, (or 30 minutes) filtered and washed with 2 volume ethanol. And dried in vacuum oven at 55° C.

Dry weight—10 g

1. A crystalline polymorph 1A of dolutegravir sodium having an X-ray powder diffraction pattern comprising peaks, in terms of degrees 2-theta, at 15.66°, 20.29° and 22.51°±0.2° 2 theta.

2. The crystalline polymorph 1A of dolutegravir sodium according to claim 1 having an X-ray powder diffraction pattern comprising further peaks, in terms of degrees 2-theta, at 8.00° and 25.53°±0.2° 2 theta.

3. The crystalline polymorph 1A of dolutegravir sodium according to claim 1 having an X-ray powder diffraction pattern comprising further peaks, in terms of degrees 2-theta, at 18.36°, 21.11°, and 23.49°±0.2° 2 theta.

4. The crystalline polymorph 1A of dolutegravir sodium according to claim 1 characterised by an X-ray powder diffraction pattern substantially as depicted in FIG. 1.

5. The crystalline polymorph 1A of dolutegravir sodium according to claim 1 characterised by an infrared spectrum substantially as depicted in FIG. 3.

6. The process for preparing crystalline polymorph 1A of dolutegravir sodium as defined in claim 1, the process comprising the steps of:
a) preparing a mixture of dolutegravir base with methanol,
b) cooling the reaction mixture from step a) to a temperature ranging from −10° C. to 20° C.,
c) preparing a solution of sodium hydroxide in a solvent,
d) mixing the solutions from steps b) and c) over a period of time less than 60 minutes at a temperature ranging from −10° C. to 20° C.,
e) maintaining the reaction mass from step d) at a temperature in the range of −10° C. to 20° C. over a period of time ranging from 30 minutes to 3 hours, and
f) isolating crystalline polymorph 1A of dolutegravir sodium.

7. The process according to claim 6, wherein the temperature in step a) ranges from 20° C. to 35° C.

8. The process according to claim 6, wherein the temperature in step b) ranges from −10° C. to 10° C.

9. The process according to claim 6, wherein the amount of sodium hydroxide used ranges from 1.1 to 2.1 moles with respect to starting material that is dolutegravir base or dolutegravir sodium or hydroxyprotected dolutegravir.

10. The process according to claim 6, wherein the period of time in step d) is less than 30 minutes.

11. The process according to claim 6, wherein the temperature in step d) ranges from −10° C. to 10° C.

12. The process according to claim 6, wherein the temperature in step e) ranges from −10° C. to 10° C.

13. The process according to claim 6, wherein the dolutegravir base used in step a) is formed in situ by a process that comprises the steps of:
1) dissolving crystalline dolutegravir sodium in a solvent, at a temperature ranging from 20° C. to 35° C.,
2) adding an acid to the reaction mass of step 1), and
3) extracting dolutegravir base in a solvent.

14. The process according to claim 13 wherein the temperature in step 1) ranges from 25° C. to 30° C.

15. The process according to claim 13 wherein the acid in step 2) is selected from hydrochloric acid, sulphuric acid, acetic acid, fumaric acid and citric acid.

16-25. (canceled)

26. Crystalline form 1A of dolutegravir sodium prepared according to claim 6.

27. (canceled)

28. The pharmaceutical composition comprising crystalline Form 1A of dolutegravir sodium as defined in claim 1 together with one or more pharmaceutically acceptable excipients and optionally one or more further active pharmaceutical ingredients.

29. (canceled)

30. The pharmaceutical composition comprising crystalline Form 1A of dolutegravir sodium as defined in claim 1 in combination with one or more further active pharmaceutical ingredients together with one or more pharmaceutically acceptable excipients.

31. (canceled)

32. The pharmaceutical composition according to claim 28, wherein the dosage form is selected from a list consisting of tablets, capsules, pills, caplets, lozenges, dispersible granules, dry powder syrup, ready to use suspension, parenteral dosage forms, various inhalation formulations, and transdermal formulations.

33. A method of treating HIV infection, AIDS and related conditions in a patient, said method comprising administering to the patient a pharmaceutical formulation comprising a therapeutically effective amount of crystalline Form 1A of dolutegravir sodium as defined in claim 1.

34-40. (canceled)
